(19) United States
(12) Patent Application Publication
(10) Pub. No.: US 2023/0053916 A1
(43) Pub. Date: Feb. 23, 2023

(54) METHODS AND SYSTEMS FOR NUCLEIC ACID SYNTHESIS

(71) Applicants: Replay Therapeutics, Inc., San Diego, CA (US); Neochromosome, Inc., Long Island City, NY (US)

(72) Inventors: Leslie MITCHELL, San Francisco, CA (US); Adrian WOOLFSON, San Francisco, CA (US)

(21) Appl. No.: 17/550,165

(22) Filed: Dec. 14, 2021

Related U.S. Application Data
(63) Continuation of application No. PCT/US2021/048675, filed on Sep. 1, 2021.
(60) Provisional application No. 63/073,389, filed on Sep. 1, 2020.

(51) Int. Cl. C12Q 1/6834

(52) U.S. Cl. CPC C12Q 1/6834; C12Q 2525/161

(57) ABSTRACT

The present disclosure provides methods, compounds, and systems for synthesizing a nucleic acid molecule. The methods may comprise spatially separating a set of barcoded oligonucleotides corresponding to a target nucleic acid molecule and performing a nucleic acid assembly using the oligonucleotides. A computer system coupled to a process control software program may be configured to apparatuses and configured to carry out the methods of the present disclosure.

### CROSS-REFERENCE TO RELATED APPLICATIONS

This application is a continuation of International Application PCT/US2021/48675, filed Sep. 1, 2021, which claims the benefit of U.S. Provisional Patent Application No. 63/073,389 filed Sep. 1, 2020 which is herein incorporated by reference in its entirety for all purposes.

### BACKGROUND

Homology-based nucleic acid assembly methods such as polymerase cycling assembly and Gibson assembly are routinely used to synthesize large DNA polynucleotides. However, such homology-based methods suffer from unexpected homology between fragments and are thus particularly unsuited to the assembly of nucleic acids with highly repetitive sequences. Beyond accuracy there is also a need to balance scalability, automation, speed, and cost. Thus, there remains a need for quick, accurate, and cost-effective methods of nucleic acid assembly.

### SUMMARY

Provided herein are methods, compositions, and systems for synthesis of polynucleotides, including assembly of extremely large polynucleotides (e.g., entire genes or genomes) and/or polynucleotides with one or more homopolymeric regions.

An aspect of the present disclosure provides for a method of generating a target polynucleotide, the method comprising: (a) providing a nucleic acid molecule comprising a stem-loop and a barcode sequence; (b) contacting a solid support with the nucleic acid molecule, wherein the solid support comprises a capture sequence complementary to the barcode sequence, thereby forming a capture complex comprising the capture sequence and the nucleic acid molecule; (c) separating the nucleic acid molecule from the capture complex; and (d) incubating the nucleic acid molecule with assembly reagents, thereby generating at least a portion of the target polynucleotide.

In some embodiments, the solid support comprises a bead. In some embodiments, the solid support comprises a microwell. In some embodiments, the microwell is on a printed array. In some embodiments, the nucleic acid molecule further comprises a restriction enzyme sequence. In some embodiments, the barcode sequence is positioned 5′ of the restriction enzyme sequence. In some embodiments, the assembly reagents comprise a polymerase, a ligase, a restriction enzyme, or any combination thereof. In some embodiments, the ligase is a T4 ligase. In some embodiments, restriction enzyme is a type IIS restriction enzyme. In some embodiments, the barcode sequence is interior to the stem-loop. In some embodiments, the barcode sequence is positioned 5′ of the stem-loop. In some embodiments, the barcode sequence is adjacent to the stem-loop. In some embodiments, the barcode sequence is positioned at the 5′ end of the nucleic acid molecule. In some embodiments, the barcode sequence is interior to a different stem-loop. In some embodiments, the different stem-loop is positioned 5′ of the stem-loop. In some embodiments, the barcode sequence is positioned at the 3′ end of the nucleic acid molecule. In some embodiments, the nucleic acid molecule further comprises a restriction enzyme site adjacent to the barcode sequence. In some embodiments, the nucleic acid molecules comprise a 3′ unpaired region. In some embodiments, the 3′ unpaired region comprises at least two nucleotides. In some embodiments, the nucleic acid molecule is single-stranded. In some embodiments, the separating in step (c) comprises contacting the capture complex with oligonucleotides complementary to the capture sequence. In some embodiments, the oligonucleotides comprise a nucleic acid analogue or a chemically modified nucleic acid. In some embodiments, the separating in step (c) comprises a thermal denaturation. In some embodiments, the nucleic acid molecule in step (a) is provided in a plurality of nucleic acid molecules comprising a stem-loop and a barcode sequence. In some embodiments, the plurality of nucleic acid molecules comprises one or more different barcode sequences. In some embodiments, plurality of nucleic acid molecules is contained in one reaction volume. In some embodiments, the plurality of nucleic acid molecules comprises at least two nucleic acid molecules comprising different barcode sequences, wherein the different barcode sequences define a plurality of subsets of the nucleic acid molecules. In some embodiments, the plurality of nucleic acid molecules comprises at least five nucleic acid molecules comprising different barcode sequences. In some embodiments, the plurality of nucleic acid molecules comprises at least ten nucleic acid molecules comprising different barcode sequences. In some embodiments, the plurality of nucleic acid molecules comprises at least twenty nucleic acid molecules comprising different barcode sequences. In some embodiments, the plurality of nucleic acid molecules comprises at least fifty nucleic acid molecules comprising different barcode sequences. In some embodiments, the plurality of nucleic acid molecules comprises at least one hundred nucleic acid molecules comprising different barcode sequences. In some embodiments, the target polynucleotide comprises molecules from more than one subset of the plurality of subsets of the nucleic acid molecules. In some embodiments, the target polynucleotide comprises at least 50 nucleotides. In some embodiments, the target polynucleotide comprises at least 100 nucleotides. In some embodiments, the target polynucleotide comprises at least 200 nucleotides. In some embodiments, the target polynucleotide comprises at least 300 nucleotides. In some embodiments, the target polynucleotide comprises at least 500 nucleotides. In some embodiments, the target polynucleotide comprises at least 1,000 nucleotides. In some embodiments, the target polynucleotide comprises at least 2,500 nucleotides. In some embodiments, the target polynucleotide comprises at least 5,000 nucleotides. In some embodiments, the target polynucleotide comprises at least 10,000 nucleotides. In some embodiments, the target polynucleotide comprises at least 20,000 nucleotides. In some embodiments, the target polynucleotide comprises at least 50,000 nucleotides. In some embodiments, the target polynucleotide comprises at least 100,000 nucleotides. In some embodiments, the target polynucleotide comprises at least 150,000 nucleotides. In some embodiments, the target polynucleotide comprises at least one homopolymeric region. In some embodiments, the target polynucleotide comprises at least two homopolymeric regions. In some embodiments, the target polynucleotide comprises at least five homopolymeric regions. In some embodiments, the target polynucleotide comprises at least ten homopolymeric regions. In some embodiments, the target polynucleotide comprises at least twenty homopolymeric regions. In some embodiments, the target polynucleotide comprises at least fifty homopolymeric regions. In some embodiments, the target polynucleotide comprises at least one hundred homopolymeric regions. In some embodiments, a homopolymeric region of the target molecule is at least two bases long. In some embodiments, a homopolymeric region of the target molecule is at least twenty-five bases long. In some embodiments, a homopolymeric region of the target molecule is at least fifty bases long. In some embodiments, a homopolymeric region of the target molecule is at least one hundred bases long. In some embodiments, a homopolymeric region of the target molecule is at least two hundred bases long. In some embodiments, a homopolymeric region of the target molecule is at least three hundred bases long. In some embodiments, a homopolymeric region of the target molecule is at least four hundred bases long. In some embodiments, a homopolymeric region of the target molecule is at least five hundred bases long. In some embodiments, the target polynucleotide comprises a deoxyribonucleic acid (DNA) molecule. In some embodiments, the target polynucleotide is a gene. In some embodiments, the method further comprises repeating steps (a)-(d) to form a genome of an organism. In some embodiments, the organism is a bacterium. In some embodiments, the organism is a fungus. In some embodiments, the organism is a virus. In some embodiments, the organism is an archaeon. In some embodiments, the organism is an alga. In some embodiments, the organism is a protist. In some embodiments, the organism is a multi-cellular organism. In some embodiments, the target polynucleotide is a regulatory element. In some embodiments, the target polynucleotide comprises a ribonucleic acid (RNA) molecule. In some embodiments, incubation is performed in a droplet comprising the bead. In some embodiments, the method further comprises breaking or disrupting the droplet. In some embodiments, the incubating in (d) takes place in the microwell. In some cases, the method further comprises subjecting the at least the portion of the target polynucleotide to amplification to generate one or more copies of the at least the portion of the target polynucleotide. In some embodiments, the incubating is carried out isothermally.

Another aspect of the present disclosure provides for a method of generating a target nucleic acid sequence comprising the steps of: (a) localizing a plurality of oligonucleotide subsequences defining an oligonucleotide set corresponding to a particular target nucleic acid sequence by hybridization to a capture sequence attached to a solid support that is unique to each oligonucleotide set, wherein an oligonucleotide subsequence of the plurality of oligonucleotide subsequences comprises a barcode sequence at a position, and wherein the barcode sequence is specific to the capture sequence; (b) contacting the solid support with a sample comprising the oligonucleotide set corresponding to the particular target nucleic acid sequence under conditions sufficient to facilitate binding between the capture sequence and the barcode sequence; (c) separating the one or more bound oligonucleotides from the solid support to generate one or more pooled oligonucleotides; and (d) generating one or more of the target nucleic acid molecule from the one or more pooled oligonucleotides.

In some embodiments, the solid support is a microwell. In some embodiments, the microwell is comprised on an array. In some embodiments, the solid support is a bead. In some embodiments, the position is at the 5′ end of the oligonucleotide subsequence. In some embodiments, more than one nucleic acid bases at the 3′ end of the barcode sequence comprises a mismatch with at least the last 2 bases at the 3′ end of the oligonucleotide subsequence. In some embodiments, the position is within a 5′ localized stem loop of the oligonucleotide subsequence. In some embodiments, the position is adjacent to a type IIS restriction site of the oligonucleotide subsequence. In some embodiments, the position is within a stem loop encoded near the 5′ stem loop of the oligonucleotide subsequence.

Another aspect of the present disclosure provides for an oligonucleotide comprising a barcode sequence at a position, wherein the position: is within a 5′ localized stem loop of the oligonucleotide subsequence; is adjacent to a type IIS restriction site of the oligonucleotide; or is within a stem loop encoded near the 5′ stem loop of the oligonucleotide.

In some embodiments, more than one nucleic acid bases at the 3′ end of the barcode sequence comprises a mismatch with at least the last 2 bases at the 3′ end of the oligonucleotide.

Another aspect of the present disclosure provides for a method for synthesizing a polynucleotide comprising: assembling a plurality of nucleic acid sequence to yield the polynucleotide at an accuracy of at least 85% in at most 60 minutes, which polynucleotide has a length of at least 500 base pairs (bp).

In some embodiments, the polynucleotide comprises at least 1,000 bp. In some embodiments, the polynucleotide comprises at least 2,500 bp. In some embodiments, the polynucleotide comprises at least 5,000 bp. In some embodiments, the polynucleotide comprises at least 10,000 bp. In some embodiments, the polynucleotide comprises at least 20,000 bp. In some embodiments, the polynucleotide comprises at least 50,000 bp. In some embodiments, the polynucleotide comprises at least 100,000 bp. In some embodiments, the polynucleotide comprises at least 150,000 bp. In some embodiments, the target polynucleotide comprises at least one homopolymeric region. In some embodiments, the polynucleotide comprises at least two homopolymeric regions. In some embodiments, the polynucleotide comprises at least five homopolymeric regions. In some embodiments, the polynucleotide comprises at least ten homopolymeric regions. In some embodiments, the polynucleotide comprises at least twenty homopolymeric regions. In some embodiments, the polynucleotide comprises at least fifty homopolymeric regions. In some embodiments, the polynucleotide comprises at least one hundred homopolymeric regions. In some embodiments, the accuracy is at least 90%. In some embodiments, the accuracy is at least 95%. In some embodiments, the accuracy is at least 98%. In some embodiments, the accuracy is at least 99%. In some embodiments, the accuracy is determined by sequencing the polynucleotide. In some embodiments, the accuracy is determined by performing as assay corresponding to a transcription or translation product of the polynucleotide.

Additional aspects and advantages of the present disclosure will become readily apparent to those skilled in this art from the following detailed description, wherein only illustrative cases of the present disclosure are shown and described. As will be realized, the present disclosure is capable of other and different cases, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative cases, in which the principles of the invention are utilized, and the accompanying drawings (also “Figure” and “FIG.” herein), of which:

FIG. 1 shows a method of extending a nucleic acid molecule.

FIGS. 2A-2C show various embodiments of the barcode sequences as described herein.

FIG. 2A shows an embodiment where the barcode sequence is at the 5′ end. FIG. 2B shows an embodiment where the barcode sequence is between the stem and the type IIS restriction site.

FIG. 2C shows an embodiment where the barcode sequence is within a stem loop encoded near the 5′ end of the nucleic acid molecule.

FIGS. 3A-3C show a flow chart of an embodiment of the methods and systems described herein. FIG. 3A shows capturing oligonucleotides comprising specific barcode sequences from an oligonucleotide pool using complimentary capture sequences to enable oligo extension. FIGS. 3B and 3C show examples of solid supports.

FIG. 4 shows a computer system that is programmed or otherwise configured to implement methods provided herein.

FIG. 5 shows capturing oligonucleotides comprising a specific barcode sequence at the 3′ of the oligonucleotide.

FIG. 6 illustrates a process for determining sequences for oligonucleotides.

### DETAILED DESCRIPTION

While various cases of the invention have been shown and described herein, it will be obvious to those skilled in the art that such cases are provided by way of example only. Numerous variations, changes, and substitutions may occur to those skilled in the art without departing from the invention. It should be understood that various alternatives to the cases of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “nucleotide,” as used herein, generally refers a molecule that can serve as the monomer, or subunit, of a nucleic acid, such as deoxyribonucleic acid (DNA) or ribonucleic acid (RNA), or analog thereof. Non-limiting examples of nucleotides include adenosine (A), cytosine (C), guanine (G), thymine (T), uracil (U), and variants thereof. A nucleotide can include any subunit that can be incorporated into a growing nucleic acid strand. A nucleotide may be a modified nucleotide, such as a locked nucleic acid (LNA). A nucleotide may be unlabeled or labeled with one or more tags. A labeled nucleotide may yield a detectable signal, such as an optical signal, electrical signal, chemical signal, mechanical signal, or combinations thereof. A nucleotide can be a deoxynucleotide (dNTP) or an analog thereof, e.g., a molecule having one or more phosphates in a phosphate chain, such as at least 1, 2, 3, 4, 5, 6, 7, 8, 9, or 10 phosphates. A nucleotide can be a dideoxynucleotide (ddNTP). Dideoxynucleotides (ddNTPs), unlike dNTPs, generally lack both 2′ and 3′ hydroxyl groups and, after being added to a growing nucleotide chain, can result in chain termination.

As used herein, the terms “polynucleotide”, “oligonucleotide”, “nucleic acid” and “nucleic acid molecule” generally refer to a polymeric form of nucleotides (polynucleotide) of various lengths (e.g., at least 2, 3, 4, 5, 6, 7, 8, 9, 10, 20, 30, 40, 50, 100, 1,000, 10,000, 100,000, 1,000,000, 10,000,000, 100,000,000 nucleotides or longer), either ribonucleotides, deoxyribonucleotides, or analogs thereof. This term may refer to the primary structure of the molecule. Thus, the term may include triple-, double- and single-stranded DNA, as well as triple-, double- and single-stranded RNA. Non-limiting examples of polynucleotides include coding and non-coding regions of a gene or gene fragment, intergenic DNA, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), small nucleolar RNA, ribozymes, complementary DNA (cDNA); DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, 2′OMe modified nucleotides and nucleotide analogs, and 2′-fluoro modified nucleotides and nucleotide analogs. If present, modifications may be imparted before or after assembly of the polymer. Nucleic acids can comprise phosphodiester bonds (e.g., natural nucleic acids). Nucleic acids can comprise nucleic acid analogs that may have alternate backbones, comprising, for example, phosphoramide (see, e.g., Beaucage et al., Tetrahedron (1993) 49(10):1925 and U.S. Pat. No. 5,644,048), phosphorodithioate (see, e.g., Briu et al., J. Am. Chem. Soc. (1989) 11 1:2321), O-methylphosphoroamidite linkages (see, e.g., Eckstein, Oligonucleotides and Analogues: A Practical Approach, Oxford University Press), and peptide nucleic acid (PNA) backbones and linkages (see, e.g., Carlsson et al., Nature (1996) 380:207). Nucleic acids can comprise other analog nucleic acids including those with positive backbones (see, e.g., Denpcy et al., Proc. Natl. Acad. Sci. (1995) 92:6097); non-ionic backbones (see, e.g., U.S. Pat. Nos. 5,386,023, 5,637,684, 5,602,240, 5,216,141 and 4,469,863; Kiedrowshi et al., Angew. Chem. Intl. Ed. English (1991) 30:423; Letsinger et al., J. Am. Chem. Soc. (1988) 110:4470; Letsinger et al., Nucleoside & Nucleotide (1994) 13:1597; Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. (1994) 4:395; Jeffs et al., J. Biomolecular NMR (1994) 34:17; Horn T., et al.,Tetrahedron Lett. (1996) 37:743); and non-ribose backbones, (see, e.g., U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook). Nucleic acids can comprise one or more carbocyclic sugars (see, e.g., Jenkins et al., Chem. Soc. Rev. (1995) pp 169-176). These modifications of the ribose-phosphate backbone can facilitate the addition of labels or increase the stability and half-life of such molecules in physiological environments.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Polynucleotides

The present disclosure provides methods, compositions, and systems for the production of polynucleotides. A polynucleotide may be single-stranded, double stranded, or inclusive of single-stranded and double stranded regions. A polynucleotide of the present invention may be a polynucleotide having a sequence of interest or a variant of a polynucleotide having a sequence of interest. A polynucleotide may further include additional nucleotides located 3′ or 5′ of the sequence of interest on one or both strands. The polynucleotide may have DNA nucleobases, RNA nucleobases, modified RNA or DNA nucleobases, synthetic or artificial nucleobases, or a mixture thereof. In some cases, the polynucleotide has only DNA nucleobases. A polynucleotide may be a specific sequence produced, or intended to be produced, in a method of assembling nucleotides. A polynucleotide may include nicks or gaps, provided that the nucleobases of the polynucleotide form a contiguous nucleic acid molecule. A polynucleotide of the present invention may be about 50 bp to 10,000 kb, or more, in length. For example, a polynucleotide may be at least about 50 bp, 100 bp, 200 bp, 300 bp, 500 bp, 1000 bp, 2,500 bp, 5,000 bp, 10 kb, 20 kb, 50 kb, 100 kb, 150 kb, 200 kb, 300 kb, 500 kb, 1,000 kb, 2,500 kb, 5,000 kb, 10,000 kb or more in length.

In some cases, the assembled polynucleotide may be a double-stranded DNA molecule. In some cases, the assembled polynucleotide may comprise both double-stranded and single-stranded segments of DNA. In some cases, the double-stranded and single-stranded segments of DNA may alternate one or more times along the length of the assembled polynucleotide. The polynucleotide may include nicks. In some cases, the polynucleotide may comprise RNA.

The polynucleotide may include introns, exons, structural sequences, or non-coding regions (e.g., untranslated regions). The polynucleotide may be a gene or gene fragment. It may encode a polypeptide, protein, enzyme, or antibody. A polynucleotide may have a sequence present in the genome of an organism. A polynucleotide may be a variant of a sequence present in the genome of an organism. A polynucleotide may include an entire genome of an organism. The organism may be a eukaryote, prokaryote, or archaea. The organism may be a fungus (e.g., a pathogen, a yeast), bacterium, virus, protist, alga, plant (e.g., a crop plant), or animal. A polynucleotide may be an artificial sequence that is not normally present in nature. The polynucleotide may comprise a barcode for various applications (e.g., a sequencing application). The sequencing may be DNA, RNA, or peptide (e.g., protein sequencing).

The polynucleotide may comprise one or more homopolymeric regions. A homopolymeric region may comprise highly repetitive, homopolymeric sequences. A homopolymeric sequence comprises a stretch of identical nucleotides, such as an adenine nucleotide sequence (poly(A)), a cytosine nucleotide sequence (poly(C)), a guanine nucleotide sequence (poly(G)), a thymine nucleotide sequence (poly(T)), an identical modified nucleotide sequence, or a sequence of identical nucleotide analogs. A homopolymeric sequence may consist of a stretch of substantially identical nucleotides with occasional substitutions of another nucleotide (e.g., a substantially poly(A) sequence with occasional guanine nucleotides). A homopolymeric sequence may comprise at least 30%, 40%, 50%, 60%, 70%, 80%, 85%, 86%, 87%, 88%, 89%, 90%, 91%, 92%, 93%, 94%, 95%, 96%, 97%, 98%, 99%, or 100% of a single nucleotide with the balance being other nucleotides. In some cases, a homopolymeric sequences may comprise not more than 100%, 99%, 98%, 97%, 96%, 95%, 94%, 93%, 92%, 91%, 90%, 89%, 88%, 87%, 86%, 85%, 80%, 70%, 60%, 50%, 40%, or 30% of a single nucleotide with the balance being other nucleotides. A homopolymeric sequence may vary in length from 2 to 1000, or more, identical nucleotides. A homopolymeric sequence may comprise a stretch of at least 2, 10, 20, 50, 100, 200, 300, 400, 500, 600, 800, 900, 1,000, or more identical nucleotides. The polynucleotide may comprise from 1 to 10,000, or more homopolymeric regions. In some cases, the polynucleotide may comprise 1, 2, 5, 10, 20, 50, 100, 150, 200, 250, 300, 350, 400, 450, 500, 600, 700, 800, 900, 1,000, 2,000, 5,000, 10,000, or more homopolymeric regions. Alternatively or additionally, the number of homopolymeric regions in a polynucleotide may be defined as a frequency of occurrence within the polynucleotide sequence. For example, a homopolymeric region may occur, on average, once every 10 bp, every 20 pb, every 50 pb, every 100 pb, every 200 bp, every 500 bp, every 1,000 bp, every 2,500 bp, every 5,000 bp, every 10 kb, every 20 kb, every 50 kb, every 100 kb, every 150 kb, every 200 kb, every 300 kb, every 500 kb, every 1,000 kb, every 2,500 kb, every 5,000 kb, or every 10,000 kb of the target polynucleotide, or more or less frequently.

### Nucleic Acid Molecules

Nucleic acid molecules may be designed to correspond to a polynucleotide of interest (e.g., a target polynucleotide) or to a variant thereof. For example, a sequence or subsequence of a nucleic acid molecule may correspond to a sequence or subsequence of a target polynucleotide. A nucleic acid molecule may include a barcode sequence, one or more cleavage sites, and/or additional nucleotides. The barcode sequence may be configured to be complementary to a capture sequence and vice versa. The capture sequence may be associated with a solid support. A nucleic acid molecule may include additional nucleotides beyond those described above or that act as a spacer.

A nucleic acid molecule may be single-stranded or double-stranded. In some cases, a nucleic acid molecule includes both double-stranded and single-stranded segments. In some cases, nucleic acid molecules are configured to form certain secondary or tertiary structures. The secondary or tertiary structures may include helical stacks, hairpins or stem-loops, multi-way junctions (e.g., 3-way or 4-way junctions), multiloops, bulged nucleotides, mismatched nucleotides, overhangs, internal loops, pseudoknots, or any combination thereof. The nucleic acid molecules may be configured to form secondary or tertiary structures at defined points in a sequence (e.g., at a 3′ or 5′ end, 3′ or 5′ of a specific sequence, 3′ or 5′ with respect to another secondary or tertiary structure).

Nucleic acid molecules may be any appropriate length. For example, the length of a nucleic acid molecule may be 20 to 2,000 or more nucleotides. In some cases, the length of a nucleic acid molecule may be 20, 50, 100, 500, 1,000, 2,000, or more nucleotides. A double-stranded nucleic acid molecule may refer to a fully double-stranded nucleic acid molecule or a double-stranded nucleic acid molecule with one or two single-stranded overhangs. The single-stranded overhangs may comprise a 3′ or a 5′ unpaired region. An unpaired region may comprise any appropriate number of bases. In some cases, the unpaired region comprises 1, 2, 3, 4, 5, 6, 7, 8, 9, 10, or more bases.

Nucleic acid molecules may be partitioned in to one or more subsets of nucleic acid molecules on the basis on their sequence, chemical modifications, structural elements, or other properties. In some cases, nucleic acid molecules are separated into subsets defined by their barcode sequences. For example, a subset of nucleic acid molecules may all share the same barcode sequence. In some cases, members of a subset may share more than one distinct barcode sequence and/or more than one copy of the same barcode sequence. In some cases, members of a subset of nucleic acid molecules may all comprise a sequence corresponding to at least a subsequence of a polynucleotide of interest (e.g., a target polynucleotide). In some cases, subsets of nucleic acid molecules may be defined by the absence of certain features. For example, members of a subset of nucleic acids may not comprise a barcode sequence and/or may not comprise a sequence corresponding to at least a portion of a target nucleotide. Subsets of nucleic acid molecules may be defined by any combination of features. For example, a subset of nucleic acid molecules may be defined by all members of the subset comprising the same barcode sequence and a portion of the same target polynucleotide (though not necessarily comprising identical subsequences of the target polynucleotide). Analogously, a subset of nucleic acid molecules may be defined by comprising identical subsequences of the same target polynucleotide but different barcode sequences.

### Barcode Sequences

A nucleic acid molecule of the present disclosure may include a barcode sequence. A barcode sequence may be present in a single-stranded region of a nucleic acid molecule. For example, a barcode sequence may be present in, for example, a 3′ or 5′ unpaired region or a loop region of a stem-loop, multiloop, or internal loop.

A barcode sequence of a nucleic acid molecule may be randomly assigned or assigned from a defined pool of candidate barcode sequences. The identifying sequence of a nucleic acid molecule may be non-randomly assigned. For example, the barcode sequence of a nucleic acid may be capable of hybridizing to a capture sequence attached to a support (e.g., a bead, a microwell). Such barcode sequence can be, e.g., a sequence complementary or substantially complementary to the barcode. In some cases, the barcode sequence may include one or more nucleotides that vary or are randomly selected, while other positions of the barcode sequence may not vary or may not be non-randomly selected. In some cases, one or more nucleic acid molecules may be synthesized such that a particular barcode is associated with one or more particular target polynucleotide sequences or parts thereof in a known manner. As a result, in such arrangements, nucleic acid molecules corresponding to particular target polynucleotides (e.g., comprising a sequence or subsequence of the target polynucleotides) may be isolated or collected by isolating or collecting nucleic acid molecules having a particular barcode sequence.

Barcode sequences may be used to organize nucleic acid molecules into capture complexes. In some cases, all of the nucleic acid molecules of a subset corresponding to a particular polynucleotide may share the same barcode sequence. In this way, a single support having capture sequences of a single corresponding sequence may capture all the nucleic acid molecules of the subset. In some cases, two or more distinct subsets, each having nucleic acid molecules sharing a single distinct identifying sequence, may be present in a collection (e.g., a pool) of nucleic acid sequences, and two or more supports, each having capture oligonucleotides corresponding to only one set, may be contacted with the pool. In such an arrangement, distinct supports may isolate or collect all of the nucleic acid sequences of distinct subsets from a single pool. In other arrangements, the pool may include two or more subsets having distinct barcode sequences and the pool may be contacted with supports configured to correspond to two or more subsets. In such an arrangement a single support may isolate or collect all of the nucleic acid molecules corresponding to two or more polynucleotides. Supports corresponding to a single polynucleotide or a plurality of polynucleotides and pools of nucleic acid molecules comprising one set or a plurality of subsets may be combined in any fashion. Supports and polynucleotides may be readily synthesized for any such arrangement according to the methods of the present invention.

In some cases, nucleic acid molecules in a given set or subset do not all share the same barcode sequence. For instance, each nucleic acid molecule in a set may have a distinct barcode sequence. Alternatively, the number of barcode sequences present in a set of nucleic acid molecules may be more than one but less than the number of nucleic acid molecules in the set. In some cases, the total number of distinct barcode sequences present in a set may be 2, 3, 4, 5, 6, 7, 8, 9, 10, or more distinct identifying sequences. In some embodiments, one or more oligonucleotides may have two or more identifying sequences. Supports with corresponding capture sequences may be synthesized.

In some cases, many distinct polynucleotides, e.g., thousands, may be synthesized and many barcodes sequences, e.g., thousands, may be provided. The present disclosure provides for massively parallel synthesis of polynucleotides. For instance, the pool may include 1, 2, 5, 10, 20, 50, 100, 200, 300, 400, 500, 600, 700, 800, 900, 1,000, 2,500, 5,000, 10,000, 20,000, 30,000, 40,000, 50,000, 60,000, 70,000, 80,000, 90,000, 100,000, or more nucleic acid sequences. The sets may correspond to 1 to 100,000 or more distinct polynucleotides of interest. The pool of nucleic acid molecules may include 2, 3, 4, 5, 6, 7, 8, 9, 10, 20, 30, 40, 50, 60, 70, 80, 90, 100, 500, 1,000, 5,000, 10,000, 50,000, 100,000, or more distinct nucleic acid molecules. The nucleic acid molecules may include as many as 1, 2, 5, 10, 20, 30, 40, 50, 100, 200, 300, 400, 500, 1,000, 2,500, 5,000, 10,000, or more, sets corresponding to distinct polynucleotides. Supports comprising capture sequences corresponding to the subsets present in the pool may isolate or collect the nucleic acid molecules required to produce particular polynucleotides of interest.

In some cases, a barcode corresponds to a polynucleotide of interest (e.g., a target polynucleotide) or part thereof. In some cases, a barcode is associated with a nucleic acid molecule that corresponds to a polynucleotide of interest. In some cases, the same barcode may be associated with a plurality of nucleic acid molecules. The plurality of nucleic acid molecules may be the same or they may be different. In some cases, different barcode sequences may be associated with one nucleic acid molecule. Different barcode sequences may define a plurality of subsets of nucleic acid molecules. In some cases, a subset of nucleic acid molecules comprises one or more nucleic acid molecules with the same barcode sequence. Alternatively, a subset of nucleic acid molecules may comprise one or more nucleic acid molecules which do not have a particular barcode sequence.

A barcode sequence may be present in a nucleic acid molecule in relation to another site or structural element of the nucleic acid molecule. For example, a barcode sequence may be positioned at the 5′ end of a nucleic acid molecule, at the 3′ end of a nucleic acid molecule, 5′ or 3′ adjacent to a secondary or tertiary structural element (e.g., a stem-loop), within a structural element (e.g., within the loop region of a stem-loop), 5′ or 3′ of a cleavage site, adjacent to a cleavage site, or any combination thereof.

In some cases, the barcode sequence comprises about 10 bases to about 200 bases. In some cases, the barcode sequence comprises about 10 bases to about 20 bases, about 10 bases to about 30 bases, about 10 bases to about 40 bases, about 10 bases to about 50 bases, about 10 bases to about 60 bases, about 10 bases to about 70 bases, about 10 bases to about 80 bases, about 10 bases to about 90 bases, about 10 bases to about 100 bases, about 10 bases to about 150 bases, about 10 bases to about 200 bases, about 20 bases to about 30 bases, about 20 bases to about 40 bases, about 20 bases to about 50 bases, about 20 bases to about 60 bases, about 20 bases to about 70 bases, about 20 bases to about 80 bases, about 20 bases to about 90 bases, about 20 bases to about 100 bases, about 20 bases to about 150 bases, about 20 bases to about 200 bases, about 30 bases to about 40 bases, about 30 bases to about 50 bases, about 30 bases to about 60 bases, about 30 bases to about 70 bases, about 30 bases to about 80 bases, about 30 bases to about 90 bases, about 30 bases to about 100 bases, about 30 bases to about 150 bases, about 30 bases to about 200 bases, about 40 bases to about 50 bases, about 40 bases to about 60 bases, about 40 bases to about 70 bases, about 40 bases to about 80 bases, about 40 bases to about 90 bases, about 40 bases to about 100 bases, about 40 bases to about 150 bases, about 40 bases to about 200 bases, about 50 bases to about 60 bases, about 50 bases to about 70 bases, about 50 bases to about 80 bases, about 50 bases to about 90 bases, about 50 bases to about 100 bases, about 50 bases to about 150 bases, about 50 bases to about 200 bases, about 60 bases to about 70 bases, about 60 bases to about 80 bases, about 60 bases to about 90 bases, about 60 bases to about 100 bases, about 60 bases to about 150 bases, about 60 bases to about 200 bases, about 70 bases to about 80 bases, about 70 bases to about 90 bases, about 70 bases to about 100 bases, about 70 bases to about 150 bases, about 70 bases to about 200 bases, about 80 bases to about 90 bases, about 80 bases to about 100 bases, about 80 bases to about 150 bases, about 80 bases to about 200 bases, about 90 bases to about 100 bases, about 90 bases to about 150 bases, about 90 bases to about 200 bases, about 100 bases to about 150 bases, about 100 bases to about 200 bases, or about 150 bases to about 200 bases. In some cases, the barcode sequence comprises about 10 bases, about 20 bases, about 30 bases, about 40 bases, about 50 bases, about 60 bases, about 70 bases, about 80 bases, about 90 bases, about 100 bases, about 150 bases, or about 200 bases. In some cases, the barcode sequence comprises at least about 10 bases, about 20 bases, about 30 bases, about 40 bases, about 50 bases, about 60 bases, about 70 bases, about 80 bases, about 90 bases, about 100 bases, or about 150 bases. In some cases, the barcode sequence comprises at most about 20 bases, about 30 bases, about 40 bases, about 50 bases, about 60 bases, about 70 bases, about 80 bases, about 90 bases, about 100 bases, about 150 bases, or about 200 bases.

### Capture Sequences

Nucleic acid molecules as disclosed herein may comprise at least one capture sequence. Capture sequences may be present on a support and capable of hybridizing to a complementary barcode sequence. The support may be, for example, a bead, a microwell, a flow cell, or the like. Capture sequences may be associated with the support by any appropriate chemical, biochemical, or physical interaction (e.g., by a biotin-streptavidin interaction). A support may include 1 to 1,000,000 or more capture sequences. In some cases, a support may include 1, 2, 5, 10, 50, 100, 500, 1000, 5,000, 10,000, 50,000, 100,000, 200,000, 300,000, 400,000, 500,000, 600,000, 700,000, 800,000, 900,000, 1,000,000 or more capture sequences. In some cases, the capture sequences present on a support may include 2 to 1,000 or more distinct capture sequences. In some cases, a support may include 1, 2, 5, 10, 20, 50, 100, 500, 1,000, 2,000, 5,000, 10,000, 100,000, or more distinct capture sequences.

A support may be configured to capture a particular group (e.g., a set or subset) of nucleic acid molecules. For example, a support may have capture oligonucleotides comprising capture sequences corresponding to every member of a set of nucleic acid molecules. In some cases, a support may be synthesized to correspond to a single set of nucleic acid molecules. In some cases, a support may be synthesized to correspond to 2 or more sets of nucleic acid molecules, such as 2 to 200. For instance, a support may include capture oligonucleotides corresponding to 2, 3, 4, 5, 6, 7, 8, 9, 10, 20, 30, 40, 50, 100, 150, or 200 sets of nucleic acid molecules.

The number of sets of nucleic acid molecules a support may be configured to isolate may not be limited to the number of capture oligonucleotides that may be present on a support, as a support may comprise numerous capture oligonucleotides of a single sequence. For instance, a support may include a total of 2-100,000 capture oligonucleotides having a particular sequence.

In some cases, the number of each distinct capture sequences on a support is the same for each distinct capture sequence. In some cases, one or more capture sequences may be present on a support in greater number than one or more other capture sequences. For example, a support may be configured to include a larger number of capture sequences corresponding to a rare, difficult to capture, or critical nucleic acid molecule. In some cases, a support may have a greater number of a terminal nucleic acid molecule than of other tile oligonucleotides. In these cases, the rare, difficult to capture, or critical nucleic acid molecule or molecules may include a barcode sequence distinct from at least one other nucleic acid molecule in a set corresponding to the support.

In some cases, a capture sequence is configured to be complementary to a barcode sequence. In some cases, the capture sequence comprises about 10 bases to about 200 bases. In some cases, the capture sequence comprises about 10 bases to about 20 bases, about 10 bases to about 30 bases, about 10 bases to about 40 bases, about 10 bases to about 50 bases, about 10 bases to about 60 bases, about 10 bases to about 70 bases, about 10 bases to about 80 bases, about 10 bases to about 90 bases, about 10 bases to about 100 bases, about 10 bases to about 150 bases, about 10 bases to about 200 bases, about 20 bases to about 30 bases, about 20 bases to about 40 bases, about 20 bases to about 50 bases, about 20 bases to about 60 bases, about 20 bases to about 70 bases, about 20 bases to about 80 bases, about 20 bases to about 90 bases, about 20 bases to about 100 bases, about 20 bases to about 150 bases, about 20 bases to about 200 bases, about 30 bases to about 40 bases, about 30 bases to about 50 bases, about 30 bases to about 60 bases, about 30 bases to about 70 bases, about 30 bases to about 80 bases, about 30 bases to about 90 bases, about 30 bases to about 100 bases, about 30 bases to about 150 bases, about 30 bases to about 200 bases, about 40 bases to about 50 bases, about 40 bases to about 60 bases, about 40 bases to about 70 bases, about 40 bases to about 80 bases, about 40 bases to about 90 bases, about 40 bases to about 100 bases, about 40 bases to about 150 bases, about 40 bases to about 200 bases, about 50 bases to about 60 bases, about 50 bases to about 70 bases, about 50 bases to about 80 bases, about 50 bases to about 90 bases, about 50 bases to about 100 bases, about 50 bases to about 150 bases, about 50 bases to about 200 bases, about 60 bases to about 70 bases, about 60 bases to about 80 bases, about 60 bases to about 90 bases, about 60 bases to about 100 bases, about 60 bases to about 150 bases, about 60 bases to about 200 bases, about 70 bases to about 80 bases, about 70 bases to about 90 bases, about 70 bases to about 100 bases, about 70 bases to about 150 bases, about 70 bases to about 200 bases, about 80 bases to about 90 bases, about 80 bases to about 100 bases, about 80 bases to about 150 bases, about 80 bases to about 200 bases, about 90 bases to about 100 bases, about 90 bases to about 150 bases, about 90 bases to about 200 bases, about 100 bases to about 150 bases, about 100 bases to about 200 bases, or about 150 bases to about 200 bases. In some cases, the capture sequence comprises about 10 bases, about 20 bases, about 30 bases, about 40 bases, about 50 bases, about 60 bases, about 70 bases, about 80 bases, about 90 bases, about 100 bases, about 150 bases, or about 200 bases. In some cases, the capture sequence comprises at least about 10 bases, about 20 bases, about 30 bases, about 40 bases, about 50 bases, about 60 bases, about 70 bases, about 80 bases, about 90 bases, about 100 bases, or about 150 bases. In some cases, the capture sequence comprises at most about 20 bases, about 30 bases, about 40 bases, about 50 bases, about 60 bases, about 70 bases, about 80 bases, about 90 bases, about 100 bases, about 150 bases, or about 200 bases.

In some cases, distinct sets of nucleic acid molecules can be retrieved simultaneously from a pool using distinct barcode and capture sequences. For example, one or more capture sequences configured to bind one or more distinct barcodes may be present on or in the same solid support (e.g., a bead, microwell) or the same portion of a solid support (e.g., zone of a flow cell, microwell on a printed array). When the barcoded solid support or portion thereof is placed in contact with the pool, only those sets of nucleic acid molecules corresponding to the barcode sequence or sequences will hybridize to the same solid support or portion thereof. In this way, sets of nucleic acid molecules may be spatially separated from certain nucleic acid molecules and place in the same reaction volume as other sets of molecules for carrying out a reaction (e.g., a nucleic acid synthesis reaction).

### Cleavage Sites

A nucleic acid molecule as described herein may comprise one or more cleavage sites. A cleavage site may comprise a sequence or portion of a nucleic acid molecule which results in the cleavage of the nucleic acid molecule or a nucleic acid molecule derived from it under certain conditions. The conditions may comprise one or more reagents, chemicals, enzymes and the like.

The one or more cleavage sites may correspond to a part of a restriction site or a template thereof. For example, a cleavage site may comprise a nucleotide sequence that when hybridized to its complement gives a restriction enzyme site. The cleavage site may correspond to any type of restriction enzyme. The restriction enzyme may correspond to that of a type I, type II, type IIS, type III, type IV, or type V restriction enzyme. In some cases, cleavage sites corresponding to restriction enzymes that produce 3′ overhangs, 5′ overhangs or blunt ends may be used. In some cases, nucleic acid molecules as described herein comprise a type IIS restriction enzyme site. Type IIS enzymes are known to cut at a distance from their recognition sites ranging from 0 to 20 base pairs. Type IIS restriction enzymes include, for example, enzymes that produce a 3′ overhang, such as, for example, BsrI, BsmI, BstF5I, BsrDI, BtsI, MnlI, BciVI, HphI, MboII, EciI, Acu I, BpmI, Mme I, BsaXI, BcgI, BaeI, BfiI, TspDTI, TspGWI, Taq II, Eco57I, Eco57MI, GsuI, PpiI, and PsrI; enzymes that produce a 5′ overhang such as, for example, BsmAI, PleI, FauI, SapI, BspMI, SfaNI, HgaI, BvbI, FokI, BceAT, BsmFI, Ksp632I, Eco31I, Esp3I, AarI; and enzymes that produce a blunt end, such as, for example, MlyI and BtrI; or any other appropriate restriction enzyme known in the art.

A cleavage site may be present in a nucleic acid molecule in relation to another site or structural element of the nucleic acid molecule. For example, a cleavage site may be positioned at the 5′ end of a nucleic acid molecule, at the 3′ end of a nucleic acid molecule, 5′ or 3′ adjacent to a secondary or tertiary structural element (e.g., a stem-loop), within a structural element (e.g., within the loop region of a stem-loop), 5′ or 3′ of a barcode sequence, adjacent to a barcode sequence, or any combination thereof.

### Solid Supports

Nucleic acid molecules of the present disclosure may be provided on a solid support. Nucleic acid sequences may be synthesized on a solid support in an array format, e.g., a microarray of single stranded DNA segments synthesized in situ on a common substrate wherein each molecule is synthesized on a separate feature or location on the substrate. Arrays may be constructed, custom ordered, or purchased from a commercial vendor. Various methods for constructing arrays are well known in the art. For example, methods and techniques applicable to synthesis of construction and/or selection oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

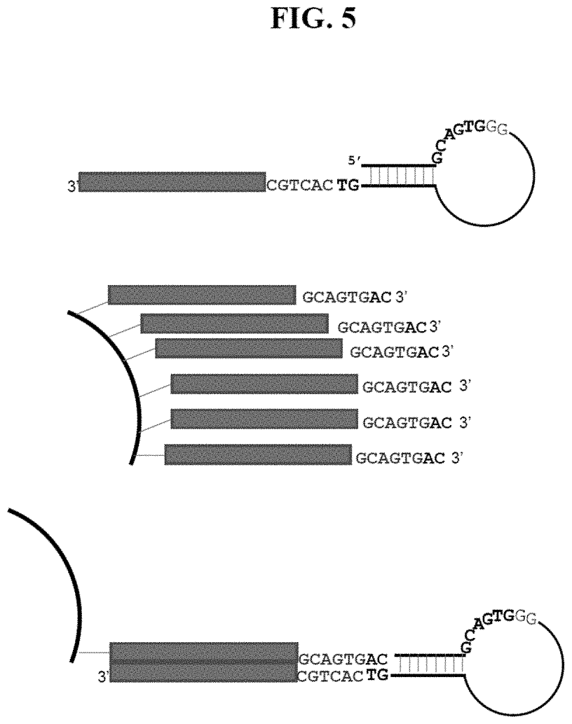

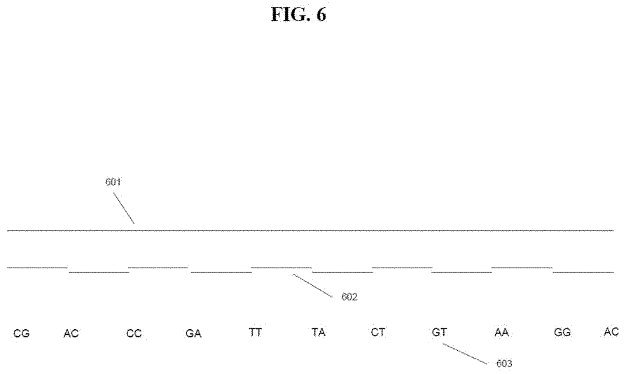

In some cases, nucleic acid molecules may be synthesized on a solid support using a maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence (See FIG. 5 of U.S. Pat. No. 6,375,903, based on the use of reflective optics). In some cases, a maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequences every day or even multiple times per day on one instrument. The differences in DNA sequence of the DNA segments in the microarray can also be slight or dramatic.

Other methods synthesizing construction and/or selection oligonucleotides include, for example, light-directed methods utilizing masks, flow channel methods, spotting methods, pin-based methods, and methods utilizing multiple supports.

Light directed methods utilizing masks (e.g., VLSIPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis of construction and/or selection oligonucleotides on a single support are described, for example, in U.S. Pat. No. 5,384,261. For example, reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support may involve delivering reactants in relatively small quantities by directly depositing them in selected regions. In some steps, the entire support surface may be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink jet printer. In some cases, the dispenser includes a series of tubes, a manifold, an array of pipettes, or the like so that various reagents may be delivered to the reaction regions simultaneously.

Pin-based methods for synthesis of oligonucleotide sequences on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microwell plate. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In some cases, a plurality of oligonucleotide sequences may be synthesized on multiple supports. One example is a bead-based synthesis method which is described, for example, in U.S. Pat. Nos. 5,770,358, 5,639,603, and 5,541,061. For the synthesis of molecules such as oligonucleotides on beads, a large plurality of beads is suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Various exemplary protecting groups useful for synthesis of oligonucleotide sequences on a solid support are described in, for example, Atherton et al., 1989, Solid Phase Peptide Synthesis, IRL Press.

Nucleic acid molecules of the present disclosure may be attached or otherwise associated with solid supports. Such supports may comprise capture sequences which hybridize to corresponding nucleic acid molecules. In some cases, solid supports include beads (e.g., Luminex microspheres, magnetic beads), chips, compartments (e.g., tubes, wells, and any other container known in the art), slides, strands, gels, sheets, spheres, capillaries, pad, slices, films, plates, and the like.

A solid support (e.g., bead, microwell, array) may have attached to it one or more capture sequences and may be used to capture nucleic acid molecules containing an identifying sequence complementary to the one or more capture sequences. A solid support or portion thereof (e.g., bead, microwell) can be attached to multiple copies of a particular capture sequence or may be attached to a plurality of distinct capture sequences. Each capture sequence on a support can, for example, hybridize to a distinct nucleic acid molecule. In some case, a solid support or portion thereof is attached to a capture sequence capable of capturing a nucleic acid containing a region corresponding to a polynucleotide of interest (e.g., a target polynucleotide). The solid support or portion thereof may, in some cases, contain multiple distinct capture sequences, each corresponding to a distinct nucleic acid molecule. The solid support or part thereof may contain multiple copies of each of these capture sequences. Thus, a bead or microwell can, for example, capture a set of nucleic acid molecules corresponding to a particular product (e.g., target polynucleotide) to be synthesized according to the methods of the invention (e.g., a gene or gene family).

### Capture Complex

A capture complex is formed when one or more capture oligonucleotides present on a support (e.g., a nucleic acid molecule comprising a capture sequence attached to a solid support) hybridize to one or more nucleic acid molecules, or a portion thereof (e.g., a barcode sequence complementary to the capture sequence). In some cases, a support may be contacted with a pool containing nucleic acid molecules of a single set. In some cases, a support may be contacted with a pool containing nucleic acids of 2 to 100,000, or more, sets corresponding to distinct polynucleotides of interest. For instance, the pool may include 2, 5, 10, 20, 50, 100, 500, 1,000, 2,000, 5,000, 10,000, 50,000, 100,000, or more sets corresponding to distinct target polynucleotides or potions thereof that may be produced by a method of the present invention in a single reaction volume. In some instances, a pool of nucleic acid molecules will be contacted with one or more supports corresponding only to a single set. In other instances, a pool of nucleic acid molecules may be contacted with 2 to 10,000 or more distinct supports corresponding to a plurality of sets. In these cases, the number of distinct supports may be 2, 5, 10, 50, 100, 500, 1,000, 5,000, 10,000, or more.

### Emulsions

An emulsion may compartmentalize or otherwise spatially separate a set of reagents or a reaction involving a set of reagents. An emulsion may comprise one or more capture complexes. In order for the nucleic acid molecules associated with a capture complex to form a corresponding polynucleotide, one, two or more, all but one, or all of the distinct nucleic acid molecules in a set may be liberated from the support. Emulsification allows nucleic acid molecules associated with a capture complex to remain isolated and co-localized when released from the capture complex for the purpose of forming a polynucleotide.

Emulsion may be achieved by a variety of methods known in the art. Methods and reagents useful in the present disclosure are described in Shendure et al., Science 309(5741):1728-32, Williams et al., Nature Methods 3:545-550 (2006), Diehl et al., Nature Methods 3:551-559 (2006), Schutze et al., Analytical Biochemistry 410:155-157 (2011), U.S. Pat. No. 10,202,628, and US Patent Publication No. 2017/0267998, each of which is incorporated herein by reference in its entirety. In some cases, the emulsion is an emulsion that is stable to a denaturing temperature, e.g., to 95° C. or higher. An emulsion may be an oil and water emulsion. In some cases, the emulsion may be a perfluorcarbon oil emulsion (e.g., a water-in-perfluorocarbon oil emulsion). A water-in-perfluorocarbon oil emulsion may be highly stable, such that the emulsion microcapsules can be stored for years with little, if any, exchange of gene products between microcapsules. Synthesis of an emulsion generally requires the application of energy (e.g., mechanical energy) to force the phases together. Methods for generating emulsions may include use of mechanical devices (e.g., stirrers, homogenizers, colloid mills, ultrasound, and membrane emulsification devices). For example, mechanical agitation can be performed using a vortex Genie. A single constituent, such as a bead can be encapsulated within an emulsion microdroplet, for example, by statistical loading, which generally involves producing an excess of emulsion microdroplets compared to the number of constituents (e.g., 10 times more microdroplets than beads). Alternatively, encapsulating single constituents (e.g., beads) within emulsion microdroplets can be achieved by making microdroplets small enough that only a single constituent can fit within each microdroplet.

An emulsion may be a well (e.g. microwell) or a plurality of wells (e.g., a plurality of microwells on a microarray) in which one or more capture complexes are compartmentalized. Compartmentalization of capture complexes into wells may be achieved, in some embodiments, due to physical limitations relating to the mass or dimensions of the capture complexes, the dimensions of the well, or a combination thereof. A well may be a fiber-optic faceplate where the central core is etched with an acid, such as an acid to which the core-cladding is resistant. A well may be a molded well. The wells may be covered to prevent communication between the wells, such that the beads present in a particular well remain within the well or are inhibited from moving into a different well. The cover may be a solid sheet or physical barrier, such as a neoprene gasket, or a liquid barrier, such as perfluorocarbon oil.

An emulsion of the present invention may be a monodisperse emulsion or heterodisperse emulsion. Each droplet in the emulsion may contain, or contain on average, 0-10 supports. For instances, a given droplet may contain 0, 1, 2, 3, 4, 5, 6, 7, 8, 9, or 10 supports. In particular embodiments, a given droplet may contain 0, 1, 2, or 3 supports. On average, the droplets of an emulsion of the present in invention may contain 0-3 supports, such as 0, 1, 2, or 3 supports, as rounded to the nearest whole number. In some embodiments, the number of supports in each emulsion droplet, on average, will be 1, or between 0 and 1, or between 1 and 2.

Emulsions as described herein may include various compounds, enzymes, or reagents in addition to the capture complex and emulsion media of the present invention. These additives may be included in the emulsion solution prior to emulsification. Alternatively, the additives may be added to individual droplets after emulsification. In some cases, additives include cleavage enzymes (e.g., restriction enzymes, e.g., Btsl), polymerases, dNTPs, ligases, competitive hybridization reagents (e.g. oligonucleotides), and other enzymes, reagents, and cofactors.

Within the emulsion droplet, one or more of the captured nucleic acid molecules may be liberated from the capture complex in order for formation of the polynucleotide to occur. In some cases, one or more tile oligonucleotides are liberated from the capture complex by thermal denaturation comprising incubation at a denaturing temperature. The denaturing temperature may be the same as or higher than an incubation temperature of a reaction to be carried out in the emulsion. The denaturing temperature may be determined by the melting temperature (Tm) of a barcode sequence and its complementary capture sequence. In some cases, the mechanism of liberation involves competitive hybridization. Oligonucleotides or analogues thereof comprising a barcode sequence are provided within the emulsion droplet. In some cases, an excess of oligonucleotides is provided such that the oligonucleotides competitively hybridize to the corresponding capture sequences and release the nucleic acid molecules. These competitive hybridization oligonucleotides may be present in an (excess) concentration sufficient to substantially replace all bound nucleic acid molecules on the capture complex. In some cases, the concentration of competitive hybridization oligonucleotides is 1, 2, 5, 10, 100, 1,000, 10,000, or more times the concentration of the corresponding nucleic acid molecule. Alternatively or additionally, the competitive hybridization oligonucleotide may comprises a nucleic acid analogue or modification (e.g., 2′-O methyl RNA, 2′-fluoro RNA, LNA, PNA) configured to bind to the corresponding capture sequence with a lower dissociation constant than the corresponding nucleic acid molecule so that the modified or analogue oligonucleotide may preferentially bind to the capture sequence and release the nucleic acid molecules from the capture complex. In some cases, the mechanism of liberation involves cleavage of the nucleic acid molecule. In these cases, one or more of the nucleic acid molecules or barcode sequences present in the capture complex may comprise a cleavage site. This cleavage site may be positioned between the support and a sequence of the nucleic acid molecule which corresponds to a sequence or subsequence of a target polynucleotide. In these cases, the emulsion may further include one or more cleavage agents capable of cleaving one or more cleavage sites present on one or more barcode sequences or captured nucleic acid molecules. In some case, the cleavage site may be an enzymatic cleavage site and the cleavage agent may be an enzyme. In some cases, the cleavage site may be a single stranded region that is cleaved by a nicking enzyme. In some cases, the cleavage site may be a restriction enzyme site. Once liberated, one or more liberated nucleic acid molecules may hybridize to one or more other nucleic acid molecules that are either similarly liberated or that remain on the support.

### Microwells

A microwell may compartmentalize or otherwise spatially separate a set of reagents or a reaction involving a set of reagents. A microwell may comprise one or more capture complexes. In some cases, the microwell is itself part of the capture complex. One or more capture sequences corresponding to one or more barcodes may be present in or on a surface of a microwell and configured to hybridize the one or more barcodes. In order for the nucleic acid molecules associated with a capture complex to form a corresponding polynucleotide, one, two or more, all but one, or all of the distinct nucleic acid molecules in a set may be liberated from the support. Localization in microwells allows nucleic acid molecules associated with a capture complex to remain isolated and co-localized when released from the capture complex for the purpose of forming a polynucleotide.

Microwells of the present disclosure may be part of a larger solid support which comprises one or more additional microwells. Supports on which a microwell may be disposed include microwell plates, chips, printed microarrays, and the like. A solid substrate may comprise any suitable number of microwells, such as 1-100,000 wells. Each well may be configured (e.g., by comprising the appropriate capture sequences) to perform the synthesis of a corresponding polynucleotide of interest or part thereof.

Microwells as described herein may include various compounds, enzymes, or reagents in addition to the capture complex and emulsion media of the present invention. These additives may be included in the emulsion solution prior to emulsification. Alternatively, the additives may be added to individual droplets after emulsification. In some cases, additives include cleavage enzymes (e.g., restriction enzymes, e.g., BtsI), polymerases, dNTPs, ligases, competitive hybridization reagents (e.g. oligonucleotides), and other enzymes, reagents, and cofactors.

Within the microwell, one or more of the captured nucleic acid molecules may be liberated from the capture complex in order for formation of the polynucleotide to occur. In some cases, one or more tile oligonucleotides are liberated from the capture complex by thermal denaturation comprising incubation at a denaturing temperature. The denaturing temperature may be the same as or higher than an incubation temperature of a reaction to be carried out in the emulsion. The denaturing temperature may be determined by the melting temperature (Tm) of a barcode sequence and its complementary capture sequence. In some cases, the mechanism of liberation involves competitive hybridization. Oligonucleotides or analogues thereof comprising a barcode sequence are provided within the emulsion droplet. In some cases, an excess of oligonucleotides is provided such that the oligonucleotides competitively hybridize to the corresponding capture sequences and release the nucleic acid molecules. These competitive hybridization oligonucleotides may be present in an (excess) concentration sufficient to substantially replace all bound nucleic acid molecules on the capture complex. In some cases, the concentration of competitive hybridization oligonucleotides is 1, 2, 5, 10, 100, 1,000, 10,000, or more times the concentration of the corresponding nucleic acid molecule. Alternatively or additionally, the competitive hybridization oligonucleotide may comprises a nucleic acid analogue or modification (e.g., 2′-O methyl RNA, 2′-fluoro RNA, LNA, PNA) configured to bind to the corresponding capture sequence with a lower dissociation constant than the corresponding nucleic acid molecule so that the modified or analogue oligonucleotide may preferentially bind to the capture sequence and release the nucleic acid molecules from the capture complex. In some cases, the mechanism of liberation involves cleavage of the nucleic acid molecule. In these cases, one or more of the nucleic acid molecules or barcode sequences present in the capture complex may comprise a cleavage site. This cleavage site may be positioned between the support and a sequence of the nucleic acid molecule which corresponds to a sequence or subsequence of a target polynucleotide. In these cases, the emulsion may further include one or more cleavage agents capable of cleaving one or more cleavage sites present on one or more barcode sequences or captured nucleic acid molecules. In some case, the cleavage site may be an enzymatic cleavage site and the cleavage agent may be an enzyme. In some cases, the cleavage site may be a single stranded region that is cleaved by a nicking enzyme. In some cases, the cleavage site may be a restriction enzyme site. Once liberated, one or more liberated nucleic acid molecules may hybridize to one or more other nucleic acid molecules that are either similarly liberated or that remain on the support.

### Methods of Synthesizing Polynucleotides

Disclosed herein are methods for synthesizing a target polynucleotide. The methods may be used to assemble polynucleotides that are long (e.g., up to hundreds of kilobases or longer) and/or contain highly repetitive regions (e.g., one or more homopolymeric regions). Nucleic acid extension reactions

Methods of the present disclosure may generally take advantage of nucleic acid extension reactions (e.g., polymerizations) known in the art. The methods and components of such reactions are described in standard treatises and texts in the field, e.g., Kornberg and Baker, DNA Replication, Second Edition (W.H. Freeman, New York, 1992); Lehninger, Biochemistry, Second Edition (Worth Publishers, New York, 1975); and the like. Providing nucleic acid molecules

The methods may comprise a step of providing a nucleic acid molecule. The nucleic acid molecule may be provided as part of an ensemble of nucleic acid molecules, such as a pool of nucleic acid molecules (e.g., an oligo pool). Oligo pools are commercially available (e.g., from Twist Biosciences of South San Francisco, CA or Agilent Technologies, Inc. of Santa Clara, Calif.) or may be produced by methods described herein or in, e.g., U.S. Pat. No. 10,202,628, which is herein incorporated by reference in its entirety.

Nucleic acid molecules in a pool of nucleic acid molecules (e.g., oligonucleotides) may comprise a sequence that corresponds to a target polynucleotide or a part thereof. The oligonucleotides may further comprise a barcode sequence and/or a cleavage site (e.g., a restriction enzyme site or part thereof). The oligonucleotides may be configured to form a particular secondary or tertiary structure. The particular secondary or tertiary structures, barcode sequence, and cleavage site may be organized with respect to one another in any suitable configuration. For example, a barcode sequence and/or cleavage site may be positioned 3′ of a cleavage site, 5′ of a cleavage site, 3′ adjacent to a cleavage site, or 5′ adjacent to a cleavage site. Alternatively or additionally, a barcode sequence and/or a cleavage site may be configured to be positioned with respect to certain structural features of an oligonucleotide. For example, a barcode and/or cleavage site may be positioned at the 5′ end of an oligonucleotide, at the 3′ end of an oligonucleotide, within a particular secondary or tertiary structure (e.g., a stem-loop) of an oligonucleotide, adjacent to a particular secondary or tertiary structure (e.g., a stem-loop) of an oligonucleotide, or some combination thereof. For example, a barcode sequence may be positioned at the 5′ end of an oligonucleotide adjacent to the helical stack of a stem-loop such that the first two bases of the barcode sequence comprise a mismatch with two bases on the 3′ end of the oligonucleotide. A cleavage site is positioned 3′ of the barcode sequence in, e.g., the loop region of the stem-loop. In another example, a barcode sequence is positioned in the loop region of a stem-loop which is itself 5′ of another stem-loop in the oligonucleotide. The nucleotide further comprises a 3′ overhang adjacent to a helical stack. A cleavage site is positioned 3′ of the barcode sequence. In still another example, a barcode sequence is positioned 5′ of a cleavage site, and both the barcode sequence and the cleavage site are positioned within the loop region of a stem-loop. A 3′ unpaired region comprising an overhang is adjacent to the stem of the stem-loop. Still further combinations of sequence and structural elements are envisaged.

### Forming Capture Complexes

Methods of synthesizing a polynucleotide may comprise a step of contacting the nucleic acid molecules with one or more solid supports to form one or more capture complexes. The one or more solid supports may comprise capture oligonucleotides (e.g., a nucleic acid molecule comprising a capture sequence attached to the solid support) configured to hybridize to one or more nucleic acid molecules, or a portion thereof (e.g., a barcode sequence complementary to the capture sequence). In some cases, a support may be contacted with a pool containing nucleic acid molecules of a single set. In some cases, a support may be contacted with a pool containing nucleic acids of 2 to 100,000, or more, sets corresponding to distinct polynucleotides of interest. For instance, the pool may include 2, 5, 10, 20, 50, 100, 500, 1,000, 2,000, 5,000, 10,000, 50,000, 100,000, or more sets corresponding to distinct target polynucleotides or potions thereof that may be produced by a method of the present invention in a single reaction volume. In some instances, a pool of nucleic acid molecules will be contacted with one or more supports corresponding only to a single set. In other instances, a pool of nucleic acid molecules may be contacted with 2 to 10,000 or more distinct supports corresponding to a plurality of sets. In these cases, the number of distinct supports may be 2, 5, 10, 50, 100, 500, 1,000, 5,000, 10,000, or more. Thus, in the methods of the present invention, a plurality of supports may be contacted with a pool containing a plurality of nucleic acid molecules, whereby each support may capture nucleic acid molecules to which it corresponds. In doing so, capture complexes are formed that collect or isolate particular sets of nucleic acid molecules.

Following isolation of one or more sets of nucleic acid molecules by formation of one or more capture complexes, the one or more capture complexes may be spatially separated from one another. In some cases, the capture complexes may already be spatially separated from one another due to the structure of the solid support (e.g., microwells in a printed array). In some cases,

Separating Nucleic Acid Molecules from Capture Complexes

Methods of synthesizing a polynucleotide may comprise a step of separating a nucleic acid molecule from the capture complex. The nucleic acid molecule may be separated from the capture complex by, for example, thermal denaturation, competitive hybridization, or digestion with a restriction enzyme. In the case of thermal denaturation, a capture complex may be heated to a certain temperature so that the bound nucleic acid molecules separate from their corresponding capture sequences. The nucleic acid molecules and/or the corresponding capture sequences may be configured such that they substantially hybridize at a low temperature (e.g., less than an incubation temperature) but substantially dissociate at a high temperature (e.g., greater than an incubation temperature). In some cases, the nucleic acid molecule may be separated from the capture complex by competitive hybridization. In such cases, the nucleic acid molecule may be separated from the capture complex by exposing the capture complex to additional oligonucleotides comprising a sequence complementary to or otherwise configured to interact with the capture sequence and displace the barcode sequence. The additional oligonucleotides may comprise DNA, RNA, nucleic acid analogues (e.g., locked nucleic acids [LNAs] or peptide nucleic acids [PNAs]), chemically modified nucleotides (e.g., 2′-O methyl or 2′-fluoro), or any combination thereof. A chemical modification may be selected on the basis of how it impacts binding affinity between an oligonucleotide and its corresponding capture sequence. In some cases, the nucleic acid molecule may be separated from the capture complex by digestion with a restriction enzyme. For example, the nucleic acid molecule may further comprise a restriction enzyme site adjacent to the barcode sequence. Upon hybridizing with the capture sequence (which is complementary to the barcode sequence and provides the other half of the restriction enzyme site), a double stranded restriction enzyme site is formed. Upon contact with the corresponding restriction enzyme, the double stranded restriction enzyme site is cleaved, liberating the oligonucleotide from the capture sequence.

### Assembly Reactions

The methods of synthesizing a polynucleotide may comprise a step of incubating the nucleic acid molecule with assembly reagents. The assembly reagents may be selected to carry out a nucleic acid assembly reaction to synthesize a target nucleic acid molecule. In some cases, the assembly reagents may comprise a polymerase (e.g., a strand displacement polymerase), a ligase, a restriction enzyme, and any combination thereof.

The assembly reagents may comprise a polymerase. A polymerase may be a transferase enzyme capable of extending a nucleotide sequence by addition of one or more nucleotides. Any suitable polymerase known in the art may be used. In some cases, the polymerase is a high fidelity polymerase. In some cases, the polymerase is a strand displacement polymerase. In some cases, the polymerase is a DNA polymerase. The DNA polymerase may be from any family of DNA polymerases including, but not limited to, Family A polymerase, Family B polymerase, Family C polymerase, Family D polymerase, Family X polymerase, and Family Y polymerase. In some instances, the DNA polymerase may be a Family B polymerase. Example Family B polymerases are from a species of, but not limited to, Pyrococcus furiosus, Thermococcus gorgonarius, Desulfurococcus strain Tok, Thermococcus sp. 9° N-7, Pyrococcus kodakaraensis, Thermococcus litoralis, Methanococcus voltae, Pyrobaculum islandicum, Archaeoglobus fulgidus, Cenarchaeaum symbiosum, Sulfolobus acidocaldarius, Bacillus virus phi29, Sulfurisphaera ohwakuensis, Sulfolobus solfataricus, Pyrodictium occultum, and Aeropyrum pernix.

Polymerases described herein for use in an assembly reaction may comprise various enzymatic activities. Polymerases are used in the methods of the invention, for example, to produce a strand complementary to a nucleic acid molecule comprising a sequence or part thereof of a target polynucleotide. In some cases, the DNA polymerase has 5′ to 3′ polymerase activity. In some cases, the DNA polymerase comprises 3′ to 5′ exonuclease activity. In other cases, the DNA polymerase does not have 3′ to 5′ exonuclease activity. In some cases, the DNA polymerase comprises proofreading activity. In other cases, the DNA polymerase does not comprise proofreading activity. Exemplary polymerases include, but are not limited to, DNA polymerase (I, II, or III), T4 DNA polymerase, T7 DNA polymerase, Bst DNA polymerase, Bca polymerase, Vent DNA polymerase, Pfu DNA polymerase, phi29 DNA polymerase, and Taq DNA polymerase. A DNA polymerase for use in the methods and systems described herein may have one of more of its enzymatic activities, such as those, described above, enhanced, reduced or eliminated by any suitable technique (e.g., site-directed mutagenesis, directed evolution).

In some cases, the assembly reagents comprise a restriction enzyme such as those discussed above. During incubation, the nucleic acid molecule may serve as a template for a nucleic acid synthesis reaction. As a result of the synthesis reaction, a restriction site is produced from a cleavage site. Once the restriction site is formed, the restriction enzyme may cleave the newly formed double-stranded nucleic acid molecule at the restriction site. Depending on the type of restriction enzyme used, cleavage by the restriction enzyme may result in a blunt end, a 3′ overhang, or a 5′ overhang. In some cases, the 3′ or 5′ overhang may be complementary to a corresponding 5′ or 3′ overhang on another nucleic acid molecule. The cleaved double-stranded nucleic acid may then hybridize with the other nucleic acid molecule.

In some cases, the assembly reagents comprise a ligase. A ligase may be an enzyme which seals “nicks” in a nucleic acid strand. For example, ligase may be an enzymatic ligation reagent or catalyst that, under appropriate conditions, forms phosphodiester bonds between the 3′-OH and the 5′-phosphate of adjacent nucleotides in DNA molecules, RNA molecules, or hybrids. In some cases, the ligase comprises bacteriophage T4 ligase, T7 ligase, and E. coli ligase, Afu ligase, Taq ligase, Tfl ligase, Tth ligase, Tth HB8 ligase, Thermus species AK16D ligase and Pfu ligase.

In some cases, an assembly reaction is performed as part of the incubation. In some cases, the incubation is substantially isothermal. In some cases, the incubation may involve one or more cycles of heating and cooling.

### Post-Assembly Treatment of Nucleic Acid Molecules

Upon completion of the assembly reaction, synthesized polynucleotides may be separated from the reaction mixture or solid support. In cases in which assembly is performed on beads in emulsions, the emulsion may be broken. The broken emulsion may include the one or more supports and any polynucleotides produced in the emulsion. In cases in which the solid support comprises a microwell or array of microwells, the solid support may be contacted with the same or another oligo pool to pull down additional oligonucleotides in the same reaction volume. A subsequent assembly step may then be carried out in the microwell or microwells. In some instances, one or more polynucleotides are attached to a support at the end of an assembly reaction. Alternatively, a polynucleotide may be free of a support. In some cases, a polynucleotide may be attached to a support but include a cleavage sequence such that the polynucleotide may be subsequently liberated from the support.

In cases in which a polynucleotide remains attached to a bead after the emulsion is broken, the polynucleotide may be isolated or collected via a detectable label present on the support. For instance, the support my include a dye label, fluorescent label, radio label, electrical conductance signal, fluorescence polarization signal, oligonucleotide label, or mass spectrometric label, or be of a particular size or shape. Examples of detectable labels further include Luminex or GnuBio labels in which ratios of squalene-type dyes or other dyes provide differentiating properties. Many detectable labels are known in the art, including many which may be present on a solid support, such as a bead (e.g., a Luminex bead). Beads useful in the methods of the invention may include differentially-dyed beads (e.g., Luminex beads) that can be analyzed by flow cytometry. Such beads can further be attached to oligonucleotide barcodes to produce barcoded beads, such as those utilized in the methods described herein. Supports may be sorted by a technique appropriate to the label or labels with which the supports are associated. Methods of sorting may include fluorescence-activated cell sorting (FACS), size separation, magnetic separation, charge separation, affinity purification, or other means known in the art. Supports isolated or collected following the breaking of the emulsion may be washed or deposited into individual wells of a microwell plate. Supports may be deposited to individual wells of a microwell plate, or each well may include a plurality of supports.

In cases in which the polynucleotide is attached to the support after the breaking of the emulsion, the base oligonucleotide may further include a cleavage site, such as a cleavage site corresponding to a cleavage reagent to which the base oligonucleotide has not been exposed. The base oligonucleotide may further be contacted with this cleavage reagent to separate the polynucleotide from the support.

### Further Rounds of Assembly

Polynucleotides generated by the methods of the present disclosure can themselves be used in a further assembly reaction. In such cases, one or more polynucleotides generated in a first round of assembly may be used as nucleic acid molecules or a starting duplex in a subsequent round of assembly. Polynucleotides for use in subsequent rounds of synthesis may be isolated or collected using capture supports, as described above. Polynucleotides for use in subsequent rounds of assembly may be separated into sets by sorting the first round supports as described above (e.g., by sorting of beads, by washing the same or another oligo pool over the solid support or supports). By these approaches, polynucleotides of virtually any length may be generated.

In some cases, following an assembly step, the products of more than one reaction volume (e.g., microwell or emulsified bead) may be pooled. Each of the pooled reaction volume or volumes may contain polynucleotides comprising subsequences of a larger target polynucleotide. The pooled reaction volumes may then be subjected to one or more additional assembly steps and subsequent rounds of pooling to synthesize longer and longer polynucleotides, eventually synthesizing a polynucleotide comprising the target sequence. Alternatively or additionally, pooled reactions may be exposed to one or more additional oligo pools to capture further subsets of nucleic acid molecules to assemble a target polynucleotide.

### Sequencing Nucleic Acids

In some cases, the methods of the present disclosure may include a step of sequencing one or more nucleic acid molecules. Nucleic acid molecule sequencing may be used to, for example, characterize a starting pool of nucleic acid molecules or to determine that a target nucleotide sequence was assembled. Any appropriate sequencing method may be used. Sequencing methods for use in methods and systems as described herein include, but are not limited to, sequencing by hybridization (SBH), sequencing by ligation (SBL), chemical sequencing, chain-termination methods (e.g., Sanger sequencing), shotgun sequencing, quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), sequencing by synthesis, ion semiconductor sequencing, nanopore sequencing, single molecule real time (SMRT) sequencing, sequencing by detecting a change in force following hybridization of an oligo. High-throughput sequencing methods, e.g., on cyclic array sequencing using platforms such as Roche 454, Illumina Solexa, AB-SOLiD, Helicos, Polonator platforms and the like, can also be utilized.

### Accuracy of Polynucleotide Synthesis

In some cases, the methods of the present disclosure are carried out to produce a product with a certain accuracy. In some cases, the accuracy of a method or product thereof may be related to an error rate. An error rate may correspond to the number of incorrectly incorporated, added, or deleted nucleotides in a target polynucleotide when aligned to the sequence of a desired target polynucleotide. For example, if a polynucleotide target comprises the sequence 5′-AAAAA-3′ and the sequence 5′-AAAAG-3′ is produced, the accuracy would be 80% with a corresponding error rate of 20%. In some cases, an accuracy of a polynucleotide synthesis may be determined by a functional assay of the target nucleotide or a downstream product thereof. For example, a target polynucleotide may comprise a gene or other sequence encoding a protein with a known function. After the target polypeptide is synthesized, the corresponding protein may be expressed from the synthesized target polynucleotide and the activity of the protein assayed. The relative activity of the protein produced from the synthesized polynucleotide as compared to a positive control may then serve as a measure of the accuracy of the synthesis process. In some cases, accuracy of polynucleotide synthesis may be evaluated by assessing the length of the assembled target nucleotide. Methods for determining the length of the assembled product are known in the art and include, for example, polyacrylamide gel electrophoresis either with or without a chemical denaturant (e.g., urea); chromatography, including gas chromatography, liquid chromatography, high-performance liquid chromatography (HPLC), affinity chromatography, ion exchange chromatography, size exclusion chromatography, expanded bed absorption chromatography, reversed-phase chromatography, hydrophobic interaction chromatography; capillary electrophoresis; and any combination thereof. In some cases, the accuracy of assembly may be assessed by measuring annealing of known probe molecules to part of the target nucleic acid molecule. Annealing of a probe may cause a signal or change in a signal such as an electrical, chemical, magnetic, mechanical, acoustical, or electromagnetic (light) signal. An electromagnetic signal may include an optical signal such as signals from fluorescence, luminescence, and absorption. In some cases, measuring the accuracy of the nucleic acid synthesis may include sequencing a product molecule or molecules. Any suitable method of sequencing, such as those discussed above, may be used.

The methods of the present disclosure may be used to synthesize a target polynucleotide with a certain accuracy. In some cases, the accuracy of the synthesis is about 30% to about 99%. In some cases, the accuracy of the synthesis is about 70%, 80%, 81%, 82%, 83%, 84%, 85%, 86%, 87%, 88%, 89%, 90%, 91%, 92%, 93%, 94%, 95,%, 96%, 97%, 98%, 99%, or more.

In some cases, a synthesis of a target polynucleotide is substantially completed in a certain time period. In some cases, the time period is about 5 min to about 1,440 min. In some cases, the time period is about 5 min to about 40 min, about 5 min to about 50 min, about 5 min to about 60 min, about 5 min to about 90 min, about 5 min to about 120 min, about 5 min to about 150 min, about 5 min to about 240 min, about 5 min to about 480 min, about 5 min to about 720 min, about 5 min to about 1,440 min, about 15 min to about 50 min, about 15 min to about 60 min, about 15 min to about 90 min, about 15 min to about 120 min, about 15 min to about 150 min, about 15 min to about 240 min, about 15 min to about 480 min, about 15 min to about 720 min, about 15 min to about 1,440 min, about 30 min to about 60 min, about 30 min to about 90 min, about 30 min to about 120 min, about 30 min to about 150 min, about 30 min to about 240 min, about 30 min to about 480 min, about 30 min to about 720 min, about 30 min to about 1,440 min, about 60 min to about 90 min, about 60 min to about 120 min, about 60 min to about 150 min, about 60 min to about 240 min, about 60 min to about 480 min, about 60 min to about 720 min, about 60 min to about 1,440 min, about 90 min to about 120 min, about 90 min to about 150 min, about 90 min to about 240 min, about 90 min to about 480 min, about 90 min to about 720 min, about 90 min to about 1,440 min, about 120 min to about 150 min, about 120 min to about 240 min, about 120 min to about 480 min, about 120 min to about 720 min, about 120 min to about 1,440 min, about 150 min to about 240 min, about 150 min to about 480 min, about 150 min to about 720 min, about 150 min to about 1,440 min, about 240 min to about 480 min, about 240 min to about 720 min, about 240 min to about 1,440 min, about 480 min to about 720 min, about 480 min to about 1,440 min, or about 720 min to about 1,440 min. In some cases, the time period is about 30 min, about 40 min, about 50 min, about 60 min, about 90 min, about 120 min, about 150 min, about 240 min, about 480 min, about 720 min, or about 1,440 min. In some cases, the time period is at least about 30 min, about 40 min, about 50 min, about 60 min, about 90 min, about 120 min, about 150 min, about 240 min, about 480 min, or about 720 min. In some cases, the time period is at most about 40 min, about 50 min, about 60 min, about 90 min, about 120 min, about 150 min, about 240 min, about 480 min, about 720 min, or about 1,440 min.

### Computer Control Systems

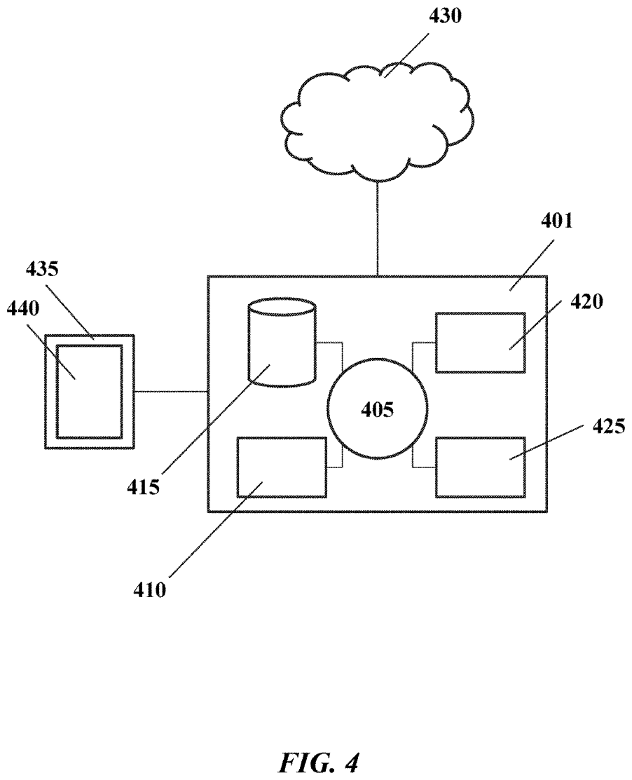

The present disclosure provides computer control systems that are programmed to implement methods of the disclosure. FIG. 4 shows a computer system 401 that is programmed or otherwise configured to carry out methods or parts of methods described herein or direct components of a system to carry out methods or parts of methods for synthesizing a target polynucleotide described herein. The computer system 401 can regulate various aspects of methods synthesizing a target polynucleotide of the present disclosure, such as, for example, designing nucleic acid molecules with defined sequences or structures for use in the methods described herein or directing a system to assembly a target polynucleotide (e.g., a gene) or plurality of target polynucleotides (e.g., a set of genes comprising a genome of an organism. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., portable PC). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 1135 that comprises a user interface (UI) 1140 for providing, for example, sequences of oligonucleotides for use in synthesizing a polynucleotide or interest or status of workflow for synthesizing a target polynucleotide. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, design oligonucleotide sequences corresponding to a target polynucleotide and configured to form certain structural features, design barcode and/or capture sequences with desired properties (e.g., length, melting temperature), initiate a micropipette assembly to carry out a process to produce a target nucleotide according to the methods of the present disclosure, etc.

### EXAMPLES

### Example 1

### Pooled Oligonucleotides as Starting Material for Nucleic Acid Assembly

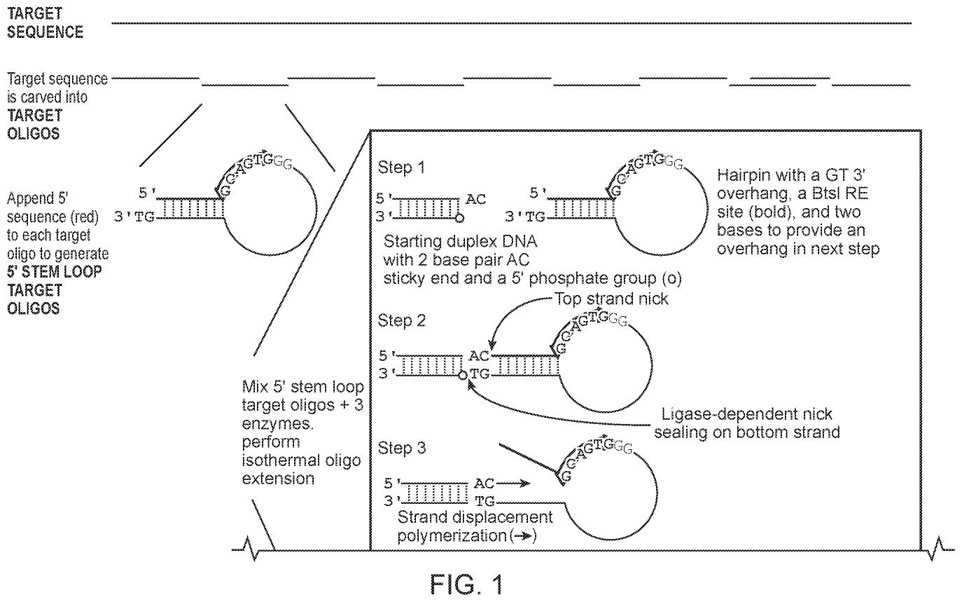

Sequence elements (“barcode sequences” as referred to herein) to enable the target-specific capture and isolation of oligonucleotides from a pool for downstream use with the nucleic acid extension methods as per FIG. 1 and CN101560538, the entirety of which is hereby incorporated by reference, are encoded onto oligonucleotide subsequences. Specifically, the oligonucleotide subsequences needed to enable such nucleic acid extension methods must: enable (i) stem loop formation and (ii) downstream IIS RE-dependent assembly through inclusion of a restriction enzyme sequence or complementary portion thereof (FIG. 1).

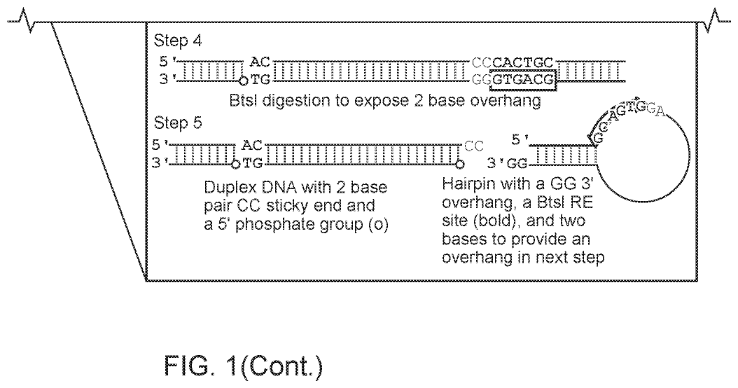

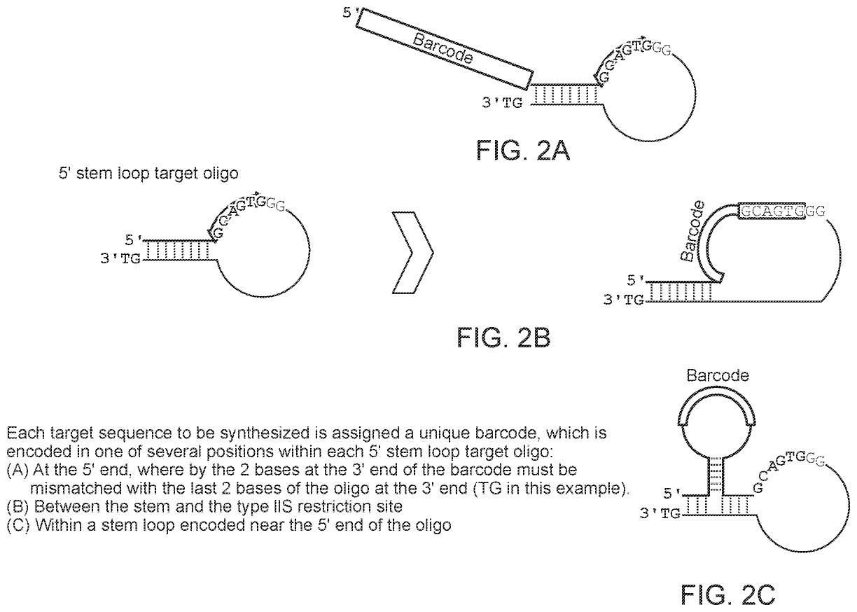

In some embodiments, the barcode sequences are encoded at the 5′ stem loop of the target oligonucleotide. One of several possible sequence formats are envisaged (FIG. 2A, B, C) for the inclusion of the barcode sequence within each oligonucleotide subsequence: at the 5′ end, where by the 2 bases at the 3′ end of the barcode must be mismatched with the last 2 bases of the oligo at the 3′ end (TG in this example) (FIG. 2A); between the stem and the type IIS restriction site (FIG. 2B); and/or within a stem loop encoded near the 5′ end of the oligonucleotide (FIG. 2C). In some embodiments, the barcode sequences are encoded at the 3′ end of the oligonucleotides (FIG. 5). In this case, an additional type IIS restriction enzyme site is positioned adjacent to the barcode sequence.

By introducing a barcode sequence of known length and sequence composition, the reverse complement sequence (e.g. “capture sequence” as used herein) can be tethered to a solid support and used to isolate the barcode-specific oligonucleotides from the pool for assembly.

### Example 2

### Selectively Retrieving Pooled Oligonucleotides

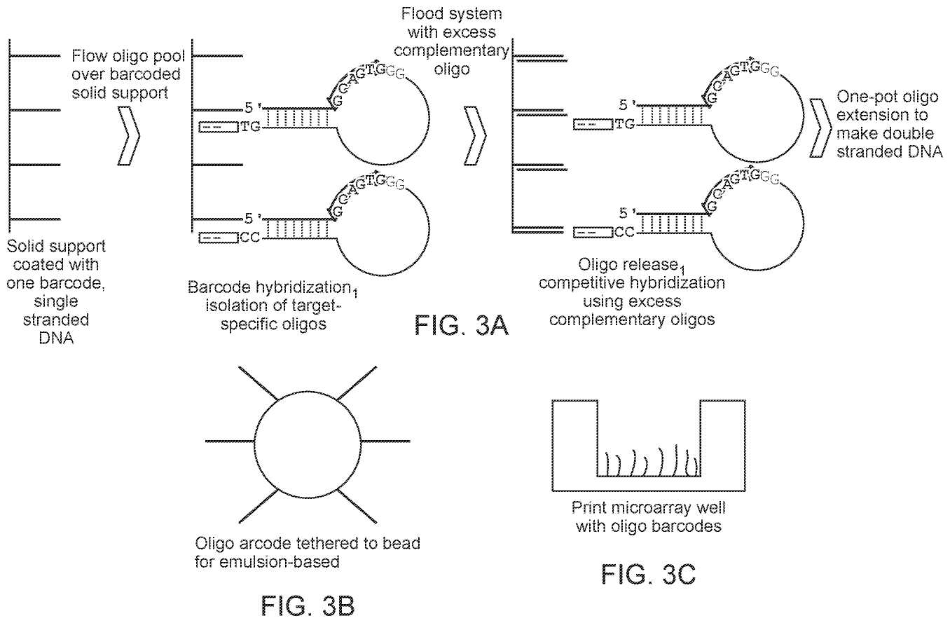

Multiple methods for executing the assembly reactions from the pool-captured oligonucleotides (FIG. 3A, B, C; FIG. 5) are envisaged. Specifically, FIG. 3A shows the use of a solid support that uses surface-specific oligonucleotides (e.g. DNA, RNA, 2′OMe modified nucleic acids, and LNA) to competitively release specific oligonucleotides from the solid support, which can then be isolated and assembled as per the oligonucleotide extension method described in FIG. 1 and CN101560538. Since the oligonucleotides have been isolated, they can be assembled in a single reaction volume to yield double-stranded DNA.

FIG. 3B shows the use of capture sequences coupled to magnetic beads that can capture specific sets of oligonucleotides comprising the complementary barcode sequence (as per Example 1) from the pool. Assembly proceeds by forming an oil-droplet emulsion for each bead, along with the required enzymes and cofactors. On heating emulsions to the required assembly temperature, captured oligonucleotides will release from the beads (bead complementary oligonucleotides may be used to assist) and assembly will occur as described in FIG. 1.

FIG. 3C shows the use of a printed array of microwells that each contain specific capture sequences (as per Example 1). The pool is washed over the array, and specific oligonucleotides are captured and spatially separated. The microarray wells are sufficiently deep that they can be used as isolated reaction vessels, with assembly occurring in a similar manner to that described in the bead-emulsion method.

FIG. 5 shows another embodiment of a solid support that uses surface-specific oligonucleotides. In FIG. 5, the barcode sequences are attached to the 3′ end of the oligonucleotides and further include a type IIS restriction enzyme site adjacent to the barcode. The capture sequence additionally has a complementary restriction enzyme sequence such that upon hybridization, a double-stranded type IIS restriction enzyme site is generated. The oligonucleotides can then be spatially separated and removed from the solid support by treatment with the type IIS restriction enzyme.

### Example 3

### Assembly of Large Nucleic Acid Molecules Comprising Highly Repetitive Regions in Emulsions

A target polynucleotide comprises a DNA sequence of thousands of base pairs (e.g., 150 kb) and at least one homopolymeric or other highly repetitive region (e.g., an HSV-1 genome). In a first step, a set of single-stranded DNA oligonucleotides corresponding to the sequence of the target polynucleotide (e.g., an oligo pool) is produced and/or sourced from a commercial vendor (e.g., Twist Biosciences [South San Francisco, Calif.], Agilent Technologies [Santa Clara, Calif.]). The oligonucleotides are configured as in Example 1 to (i) form a stem-loop, (ii) comprise a two base 3′overhang and (iii) contain a type IIS restriction enzyme site (e.g., a BtsI restriction site). The oligonucleotides further comprise barcode sequences positioned in the loop region of a stem-loop which is 5′ of the type IIS restriction enzyme site. The barcode sequences are configured to bind to a corresponding capture sequence at a relatively low temperature (e.g., below an assembly reaction temperature) and to release from the corresponding capture sequence at a relatively high temperature (e.g., at or above an assembly reaction temperature).

The sequences of the single-stranded DNA oligonucleotides are determined by a computer program comprising an algorithm which automates the process of generating a set of oligonucleotides corresponding to the target nucleotide. The process is illustrated in FIG. 6. The algorithm fragments the target sequence 601 into appropriate oligonucleotide fragments 602, appends the proper type IIS restriction enzyme site and barcode sequences, and adds the appropriate bases to generate the desired secondary structure (e.g., stem-loops and 3′ overhang 603) for each oligonucleotide. The barcode sequences are selected based on a desired melting temperature (Tm) for the barcode sequence and its complement (e.g., capture sequence) as well as minimizing nonspecific binding between noncognate barcode and capture sequence pairs.

In a second step, the oligo pool is placed in contact with a solid support or plurality of solid supports (e.g., a plurality of beads) to form a capture complex. The beads comprise capture sequences corresponding to barcode sequences on the oligonucleotides and define sets of oligonucleotides which collectively are used in a subsequent assembly step to synthesize the target polynucleotide or a portion thereof. The beads capture the sets of oligonucleotides corresponding to the barcodes on the beads. The beads are placed in an oil-droplet emulsion along with assembly reagents including enzymes, cofactors, and other reagents needed for an assembly reaction. The assembly reagents comprise a ligase (e.g., T4 DNA ligase), a type IIS restriction enzyme (e.g., BtsI), and a strand displacement polymerase.

In a third step, the emulsified beads are heated to an incubation temperature for the assembly process. Upon heating to the assembly temperature, captured oligonucleotides are released from the capture complex.

In a fourth step, the assembly process then proceeds analogously to the process described in Example 1. Briefly, an initiator comprising a starting duplex corresponding to the target polynucleotide comprising a two base sticky end (overhang) and 5′ phosphate on the anti-sense strand hybridizes with a released oligonucleotide. The two base overhang is complementary to two corresponding nucleotides on the released oligonucleotide. The initiator and released oligonucleotide hybridize, and the ligase connects the 5′ phosphate to the released oligonucleotide. The polymerase extends the strand comprising the overhang, in the process opening the stem-loop in the oligonucleotide and generating a double stranded and thus functional BtsI restriction enzyme site. The restriction enzyme site is then cut by BtsI present in the reaction mixture to cleave part of the newly synthesized double stranded DNA corresponding to the restriction enzyme site and the barcode sequence. As a result of cleavage by the BtsI, a new overhang and 5′ phosphate are generated such that the process may be repeated with the newly synthesized and cleaved double stranded DNA serving as the starting duplex. This series of steps repeats until both strands of the target polynucleotide are synthesized.

In a fifth step, steps two through four are repeated. Namely, the assembly products of the step four are pooled and subjected to further rounds of capture, release, and assembly to produce longer and longer polynucleotides. The initiator duplex may contain a barcode sequence to facilitate subsequent rounds of assembly. Eventually, the target polynucleotide is synthesized.

The assembly reaction demonstrates high fidelity as measured by sequencing and/or functional assays and, in particular, shows higher fidelity than conventional (e.g., temperature cycling and/or homology-based) assembly reactions known in the art (e.g., polymerase cycling assembly [PCA], Gibson assembly).

### Example 4

### Automated Assembly of Nucleic Acid Molecules

Systems of the present may comprise a computer with a process software program. Algorithms in the software may and control end-to-end process of apparatuses configured to carry out the methods described herein. In one example, a target polynucleotide comprising a DNA sequence of thousands of base pairs (e.g., 150 kb) and at least one homopolymeric or other highly repetitive region (e.g., an HSV-1 genome) is assembled by automation of the steps described in Example 3. The sequence of the target polynucleotide is input into a computer program comprising an algorithm, which when executed by a processor of the computer, selects a specific number of fragments and a specific order for using them to construct the target polynucleotide.

The processor is coupled to a micropipette or a plurality of micropipettes and initiates the micropipettes or plurality thereof to execute the steps of Example 3 (repeating step five of Example 3 as many times as necessary) to construct the target genome.

While preferred cases of the present invention have been shown and described herein, it will be obvious to those skilled in the art that such cases are provided by way of example only. It is not intended that the invention be limited by the specific examples provided within the specification. While the invention has been described with reference to the aforementioned specification, the descriptions and illustrations of the cases herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the cases of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-111. (canceled)

112. A method of generating a target polynucleotide, said method comprising:
(a) providing a nucleic acid molecule comprising a stem-loop and a barcode sequence;
(b) contacting a solid support with said nucleic acid molecule, wherein said solid support comprises a capture sequence complementary to said barcode sequence, thereby forming a capture complex comprising said capture sequence and said nucleic acid molecule;
(c) separating said nucleic acid molecule from said capture complex; and
(d) incubating said nucleic acid molecule with assembly reagents, thereby generating at least a portion of said target polynucleotide.

113. The method of claim 112, wherein said solid support comprises a bead.

114. The method of claim 112, wherein said solid support comprises a microwell.

115. The method of claim 114, wherein said microwell is on a printed array.

116. The method of claim 112, wherein said nucleic acid molecule further comprises a restriction enzyme sequence.

117. The method of claim 116, wherein said barcode sequence is positioned 5′ of said restriction enzyme sequence.

118. The method of claim 112, wherein said assembly reagents comprise a polymerase, a ligase, a restriction enzyme, or any combination thereof.

119. The method of claim 112, wherein said barcode sequence is interior to said stem-loop.

120. The method of claim 112, wherein said barcode sequence is positioned 5′ of said stem-loop.

121. The method of claim 120, wherein said barcode sequence is adjacent to said stem-loop.

122. The method of claim 120, wherein said barcode sequence is positioned at the 5′ end of said nucleic acid molecule.

123. The method of claim 112, wherein said barcode sequence is interior to a different stem-loop.

124. The method of claim 123, wherein said nucleic acid molecule further comprises a restriction enzyme site adjacent to said barcode sequence.

125. The method of claim 112, wherein said nucleic acid molecule comprises a 3′ unpaired region.

126. The method of claim 112, wherein said nucleic acid molecule is single-stranded.

127. The method of claim 112, wherein said separating in step (c) comprises contacting said capture complex with oligonucleotides complementary to said capture sequence.

128. The method of claim 112, wherein said separating in step (c) comprises a thermal denaturation.

129. The method of claim 112, wherein said nucleic acid molecule in step (a) is provided in a plurality of nucleic acid molecules comprising a stem-loop and a barcode sequence.

130. The method of claim 129, wherein said plurality of nucleic acid molecules comprises one or more different barcode sequences.

131. The method of claim 130, wherein said plurality of nucleic acid molecules is contained in one reaction volume.

132. The method of claim 129, wherein said plurality of nucleic acid molecules comprises at least two nucleic acid molecules comprising different barcode sequences, wherein said different barcode sequences define a plurality of subsets of said nucleic acid molecules.

133. The method of claim 112, wherein said target polynucleotide comprises at least 100,000 nucleotides.

134. The method of claim 133, wherein said target polynucleotide is a gene.

135. The method of claim 134, further comprising repeating steps (a)-(d) to form a genome of an organism.

136. The method of claim 113, wherein said incubation is performed in a droplet comprising said bead.

137. The method of claim 136, further comprising breaking or disrupting said droplet.

138. The method of claim 112, wherein said incubating is carried out isothermally.

139. An oligonucleotide comprising a barcode sequence at a position, wherein said position:
is within a 5′ localized stem loop of said oligonucleotide subsequence;
is adjacent to a type IIS restriction site of said oligonucleotide; or
is within a stem loop encoded near said 5′ stem loop of said oligonucleotide.

140. The oligonucleotide of claim 139, wherein more than one nucleic acid bases at the 3′ end of said barcode sequence comprises a mismatch with at least the last 2 bases at the 3′ end of said oligonucleotide.
